(19) United States
(12) Patent Application Publication
(10) Pub. No.: US 2024/0425488 A1
(43) Pub. Date: Dec. 26, 2024

(54) IMPROVED FOAMING BIOSURFACTANTS

(71) Applicant: LOCUS SOLUTIONS IPCO, LLC, SOLON, OH (US)

(72) Inventors: LEE SPEIGHT, SOLON, OH (US); DANIEL HAGAMAN, MECHANICSVILLE, VA (US); ANDREW MORRIS, RICHMOND, VA (US); AYENDA ZEMAITIS, MIDLOTHIAN, VA (US)

(21) Appl. No.: 18/248,441

(22) PCT Filed: Mar. 8, 2023
(86) PCT No.: PCT/US2023/014789

§ 371 Date: Apr. 10, 2023

Related U.S. Application Data
(60) Provisional application No. 63/317,583, filed on Mar. 8, 2022.

(51) Int. Cl. C07D 407/12; C07F 1/04; C07F 1/06; C07F 3/02; C07F 3/04

(52) U.S. Cl. CPC C07D 407/12; C07F 1/04; C07F 1/06; C07F 3/02; C07F 3/04

(57) ABSTRACT

The present invention provides modified sophorolipids comprising taurine for improved foaming performance. The present invention also provides compositions comprising the modified sophorolipids formulated for cosmetics and personal care products. Further provided are methods for improving skin conditions, enhancing and/or maintaining the oral health, and cleaning, enhancing and/or maintaining heathy hair of a subject.

### CROSS-REFERENCE TO RELATED APPLICATION

This application claims priority to U.S. Provisional Patent Application No. 63/317,583, filed Mar. 8, 2022, which is incorporated herein by reference in its entirety.

### BACKGROUND OF THE INVENTION

Foams are metastable dispersions of gas in a liquid matrix often stabilized by surfactant adsorption at the air-fluid interface. They find application in various industries including household and personal care, food, and environmental, oil and mineral processing. In products such as beer consumers desire stable foam while in other applications such as cleaning or processing, foaming may be controlled.

Biosurfactants are microbially-derived amphiphilic molecules consisting of both hydrophobic (e.g., a fatty acid) and hydrophilic domains (e.g., a sugar). Due to their amphiphilic nature, biosurfactants can partition at the interfaces between different fluid phases such as oil/water or water/air interfaces, reducing the surface tension of fluid and increasing the stability of bubbles, which permits the creation of abundantly steady foam.

Other excellent properties of biosurfactants, such as antimicrobial, emulsification and wettability, low toxicity, biodegradation and produced from renewable resources, allow them to be widely used in industrial production, agriculture, and daily life as wetting agents, emulsifiers, and detergents. More specifically, there is a large demand for biosurfactants for use in agriculture, environmental protection, pharmaceutical industry, food processing, oil production, the detergent industry and cosmetics.

Sophorolipids, which is one of the most promising and attractive biosurfactants that combines green chemistry with lower carbon footprint, are specific glycolipids of interest. Sophorolipids comprise a sophorose consisting of two glucose molecules, linked to a fatty acid by a glycosidic ether bond. Sophorolipids are categorized into two general forms: the lactonic form, where the carboxyl group in the fatty acid side chain and the sophorose moiety form a cyclic ester bond; and the acidic form, or linear form, where the ester bond is hydrolyzed.

Fermentation of yeast cells in a culture substrate including a sugar and/or lipids and fatty acids with carbon chains of differing length can be used to produce a variety of sophorolipids. The yeast Starmerella (Candida) bombicola is one of the most widely recognized producers of sophorolipids. Typically, the yeast produces both lactonic and linear sophorolipids during fermentation.

Sophorolipids are frequently produced as a mixture of related molecules. Differences among the related molecules arise mainly from: their fatty acid structure (degree of unsaturation, chain length, position(s) of unsaturation and position of hydroxylation); whether they are produced in the linear or lactonic form; the acetylation pattern; the presence of stereoisomers; and/or whether the glycosidic bond on the fatty acid is at the ω-position (e.g., terminal) or ω-1 position (sub-terminal).

Increasingly, consumers are looking for cleaning and personal care products that are non-toxic, non-irritating to the skin and/or eyes, and with a reduced impact on the environment, but these safer and more sustainable products are still expected to deliver performance with respect to attributes such as cleaning and foaming power.

Foaming ability and stability are key properties of surface active agents. In some industrial fields, it is often useful to add surfactants that can show certain types of surface activity with desired foaming property. Thus, there is a need for improved foaming biosurfactants for use in various applications such as personal care products that are effective and low-coast, and do not contain harmful chemicals.

### BRIEF SUMMARY OF THE INVENTION

The subject invention provides compositions exhibiting improved foaming properties. Also provided are materials and methods for producing sophorolipids that are amenable to modification; and materials and methods for modifying sophorolipids.

In certain embodiments, taurine is added covalently to a linear acidic sophorolipid through an amide bond, or to a truncated sophorolipid through an amide bond or secondary amine formation. These modified sophorolipids having improved foaming performance may be purified for formulation into, for example, beauty and personal care products.

In one embodiment, the present invention provides modified sophorolipids or salts thereof that comprise a taurine covalently added to sophorolipid via an amide bond or an amine. In some embodiments, the modified sophorolipids or salts thereof comprise a truncated fatty acid chain with a taurine covalently added to such truncated sophorolipid via an amide bond or an amine.

In specific embodiments, the compound of the subject invention has a structure selected from: i) formula (II):

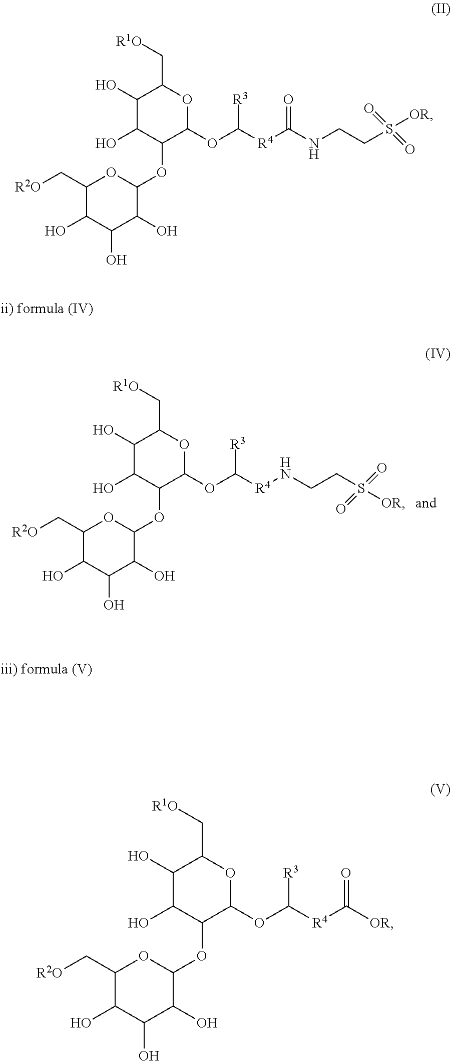

wherein R¹ and R² are each independently selected from hydrogen, alkyl, substituted alkyl, acyl, substituted acyl, alkenyl, and substituted alkenyl; R³ is hydrogen or alkyl; R⁴ is alkane, substituted alkane, alkylene, substituted alkylene, alkenylene or substituted alkenylene; and R is hydrogen or a cation selected from, for example, Na⁺, K⁺, Li⁺, Cs⁺, Rb⁺, Ca²⁺, Mg²⁺ and NH₄⁺. Preferrably, R¹ and R² are hydrogen; R⁴ is C5-C15 alkane, C5-C15 alkylene, or C5-C15 alkenylene; and R³ is methyl.

In certain embodiments, the modified sophorolipids of the subject invention can be used as active ingredients in environmentally-friendly cleaning compositions for efficiently increasing the viscosity and foam of personal care and household cleaning applications.

In one embodiment, the modified sophorolipid of the subject invention can be used as a cosmetic cleaning agent having excellent foaming and washing behavior, whilst being pleasant to use, and having advantageous biodegradability characteristics.

In one embodiment, the subject invention provides a personal care composition or a cosmetic composition, that is used for cleansing, conditioning, grooming, beautifying, or otherwise enhancing the appearance and/or well-being of a human or animal.

Personal care products include skin care products, cosmetic products, antiperspirants, deodorants, perfume, toiletries, soaps, bath oils, feminine care products, hair-care products, oral hygiene products, depilatories, shampoos, conditioners, hair straightening products, other haircare products, color cosmetics such as lipstick, creams, make-up, skin creams, lotions (preferably comprised of water-in-oil or oil-in-water emulsions), shave creams and gels, after-shave lotions and shave-conditioning compositions and sunscreen products, among numerous others. In preferred embodiments, personal care compositions according to the present invention include haircare and skin care products, especially shampoos, conditioners, rinses, detangler products, hair color products, body washes, make-up, lipstick, skin creams and other skin-care products.

In one embodiment, the subject invention provides a personal care composition comprising the modified sophorolipid of the subject invention at, for example, I to 50%, 1 to 40%, 1 to 30%, 5 to 30%, or 10 to 30% by weight.

In one embodiment, the subject invention provides a cosmetic composition comprising the modified sophorolipid of the subject invention at, for example, I to 30%, 5 to 30%, 5 to 20%, or 5 to 15% by weight.

The personal care composition of the present invention can enhance and/or facilitate deposition of an active compound onto keratinous materials, in particular the hair, comprising at least one step of applying to the keratinous material an effective amount of the composition of the present invention.

In one embodiment, the subject invention provides a body wash composition comprising the modified sophorolipid of the subject invention at, for example, 1 to 50%, 1 to 40%, 1 to 30%, 5 to 30%, or 10 to 30% by weight.

In one embodiment, the subject invention provides a shampoo composition comprising the modified sophorolipid of the subject invention at, for example, 1 to 50%, 1 to 40%, 1 to 30%, 5 to 30%, or 5 to 20% by weight.

In one embodiment, the subject invention provides a facial cleanser composition comprising the modified sophorolipid of the subject invention at, for example, 1 to 30%, 5 to 30%, 5 to 20%, or 5 to 10% by weight.

In one embodiment, the composition of the subject invention further comprises a pharmaceutically or cosmetically acceptable carrier. In one embodiment, the composition may further comprise one or more ingredients selected from viscosity modifiers, skin conditioners, anti-aging components, exfoliating agents, antioxidants, fragrances, and preservatives.

In one embodiment, the subject invention provides a method for improving the foaming performance of a sophorolipid, the method comprising providing the sophorolipid, and functionalizing the sophorolipid by covalently linking to taurine through an amide bond or amine bond.

In one embodiment, the subject invention provides a method for producing the composition of the subject invention, the method comprising providing a sophorolipid, functionalizing the sophorolipid by covalently linking to taurine through an amide bond or amine bond, and mixing the functionalized sophorolipid with a solvent.

In one embodiment, the subject invention provides a method for improving a skin condition of a subject, wherein the method comprising applying the composition of the subject invention to an area of the skin of the subject, and optionally, rinsing the composition off.

In one embodiment, the subject invention provides a method for enhancing and/or maintaining the oral health of a subject, wherein the method comprising applying the composition of the subject invention to a surface of the subject's mouth, lips or oral cavity, and optionally, rinsing the composition off.

In one embodiment, the subject invention provides a method for cleaning, enhancing and/or maintaining heathy hair of a subject, wherein the method comprising applying the composition of the subject invention to the hair of the subject, and optionally, rinsing the composition off.

### DETAILED DESCRIPTION

The subject invention provides modified sophorolipids having improved foaming performance. Such sophorolipids can be used as active ingredients in, for example, beauty and personal care (e.g., bath products, antiacne products, antidandruff products, lip cream, lipsticks, and toothpaste) products. The subject invention also provides compositions comprising a modified sophorolipid having improved foaming properties.

The subject invention also provides materials and methods for producing sophorolipids that are amenable to modification; and materials and methods for modifying sophorolipids. Specifically, taurine is added covalently to linear acidic sophorolipid through an amide bond, or to a truncated sophorolipid through an amide bond or secondary amine formation. These modified sophorolipids having improved foaming performance may be purified for formulation into beauty and personal care products (e.g., cleansing pads, deodorant, lotion, makeup, hand soap, facial cleanser, body wash, perfumes, shaving cream, moisturizer, and shampoo).

Sophorolipids are glycolipid biosurfactants produced by, for example, various yeasts of the Starmerella clade. Sophorolipids consist of a disaccharide sophorose linked to long chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid can have, for example, 11 to 20 carbon atoms, and may contain one or more unsaturated bonds. Furthermore, the sophorose residue can be acetylated on the 6- and/or 6′-position(s). The fatty acid carboxyl group can be free (acidic or linear form) or internally esterified at the 4″-position (lactonic form). In most cases, fermentation of sophorolipids results in a mixture of hydrophobic (water-insoluble) sophorolipids, including, e.g., lactonic sophorolipids, mono-acetylated linear sophorolipids and di-acetylated linear sophorolipids, and hydrophilic (water-soluble) sophorolipids, including, e.g., non-acetylated linear sophorolipids.

As used herein, the term “sophorolipid,” and “sophorolipid molecule” includes all forms, and isomers thereof, of sophorolipid molecules, including, for example, acidic (linear) sophorolipids and lactonic sophorolipids. Further included are mono-acetylated sophorolipids, di-acetylated sophorolipids, esterified sophorolipids, sophorolipids with varying hydrophobic chain lengths, sophorolipids with fatty acid-amino acid complexes attached, and other, including those that are and/or are not described within in this disclosure.

### Modified Sophorolipids

Linear sophorolipid molecules can be represented by general formula (I):

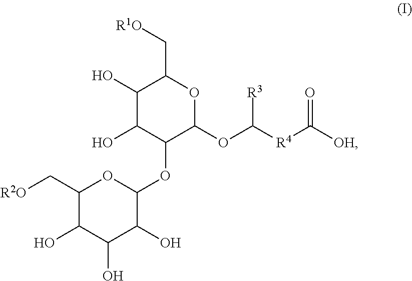

wherein R¹ and R² are each independently selected from hydrogen, alkyl, substituted alkyl, acyl, substituted acyl, alkenyl, and substituted alkenyl; R³ is hydrogen or alkyl; and R⁴ is alkane, substituted alkane, alkylene, substituted alkylene, alkenylene or substituted alkenylene. Preferrably, R¹ and R² are hydrogen; and R³ is methyl.

Sophorolipid molecules can be obtained as a collection of 30 or more types of structural homologues having different fatty acid chain lengths and degrees of unsaturation (R⁴), and, in some instances, having an acetylation or protonation at R¹ and/or R².

Examples of the substituents include halogen atoms, hydroxyl, lower (C1-6) alkyl groups, halo lower (C1-6) alkyl groups, hydroxy lower (C1-6) alkyl groups, halo lower (C1-6) alkoxy groups, and others. R⁴ typically has 11 to 20 carbon atoms. In preferred embodiments of the subject invention, R⁴ has 18 carbon atoms.

In one embodiment, the present invention provides modified sophorolipids of the general formula (II), which comprise a taurine covalently added to sophorolipid via an amide bond.

In one embodiment, the subject invention provides a compound of formula (II), which is a modified sophorolipid comprising taurine:

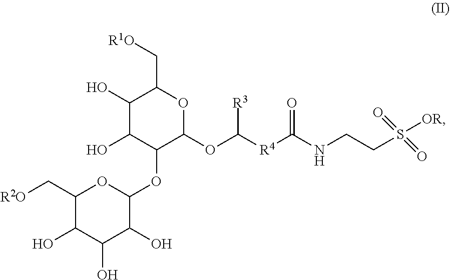

wherein R¹ and R² are each independently selected from hydrogen, alkyl, substituted alkyl, acyl, substituted acyl, alkenyl, and substituted alkenyl; R³ is hydrogen or alkyl; R⁴ is alkane, substituted alkane, alkylene, substituted alkylene, alkenylene or substituted alkenylene; and R is hydrogen or a cation selected from, for example, Na⁺, K⁺, Li⁺, Cs⁺, Rb⁺, Ca²⁺, Mg⁺ and NH₄⁺. Preferrably, R¹ and R² are hydrogen; R³ is methyl; and R⁴ is C5-C15 alkane, C5-C15 alkylene, or C5-C15 alkenylene.

In one embodiment, the compound of the subject invention is a modified sophorolipid created from a linear acidic sophorolipid covalently linked to taurine through an amide bond. In a specific embodiment, the modified sophorolipid has a structure of formula (III):

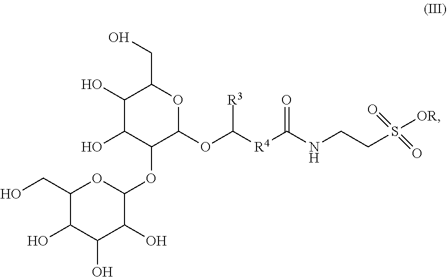

wherein R is hydrogen or a cation selected from, for example, Na⁺, K⁺, Li⁺, Cs⁺, Rb⁺, Ca²⁺, Mg²⁺ and NH₄⁺.

In a preferred embodiment, the modified sophorolipid has a structure of:

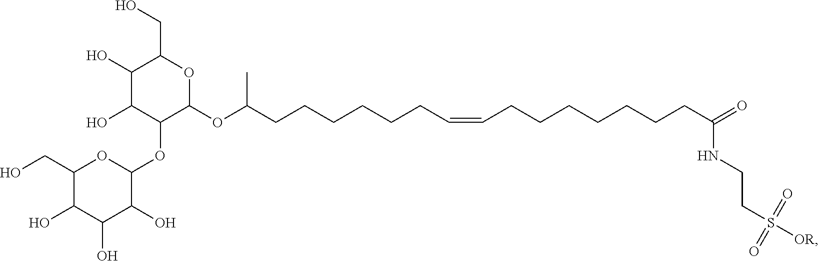

wherein R is hydrogen or a cation selected from, for example, Na⁺, K⁺, Li⁺, Cs⁺, Rb⁺, Ca²⁺, Mg²⁺ and NH₄⁺.

In one embodiment, the present invention provides modified sophorolipids of the general formula (IV), which comprise a taurine covalently added to the linear sophorolipid via an amine bond.

In one embodiment, the subject invention provides a compound of formula (IV), which is a modified sophorolipid:

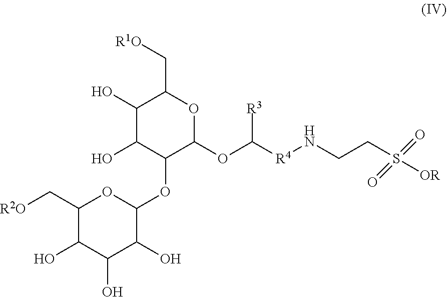

wherein R¹ and R² are each independently selected from hydrogen, alkyl, substituted alkyl, acyl, substituted acyl, alkenyl, and substituted alkenyl; R³ is hydrogen or alkyl; R⁴ is alkane, substituted alkane, alkylene, substituted alkylene, alkenylene or substituted alkenylene; and R is hydrogen or a cation selected from, for example, Na⁺, K⁺, Li⁺, Cs⁺, Rb⁺, Ca²⁺, Mg⁺ and NH₄⁺.

Preferrably, R¹ and R² are hydrogen; R³ is methyl; and R⁴ is C5-C15 alkane, C5-C15 alkylene, or C5-C15 alkenylene.

In one embodiment, the compound of the subject invention has a structure of formula (V):

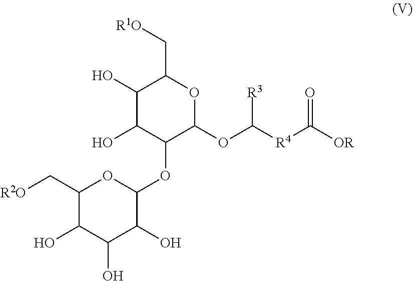

Wherein R¹ and R² are each independently selected from hydrogen, alkyl, substituted alkyl, acyl, substituted acyl, alkenyl, and substituted alkenyl; R³ is hydrogen or alkyl; R⁴ is alkane, substituted alkane, alkylene, substituted alkylene, alkenylene or substituted alkenylene; and R is hydrogen or a cation selected from, for example, Na⁺, K⁺, Li⁺, Cs⁺, Rb⁺, Ca²⁺, Mg²⁺ and NH₄⁺.

Preferrably, R¹ and R² are hydrogen; R³ is methyl and R⁴ is C5-C15 alkane, C5-C15 alkylene, or C5-C15 alkenylene.

In one embodiment, the compound of the subject invention is a modified truncated sophorolipid formed from oxidative cleavage of C18 sophorolipid or a salt thereof. In a specific embodiment, the modified sophorolipid has a structure of:

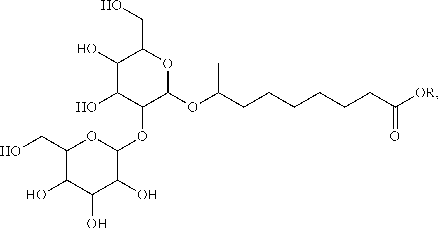

wherein R is hydrogen or a cation selected from, for example, Na⁺, K⁺, Li⁺, Cs⁺, Rb⁺, Ca²⁺, Mg²⁺ and NH₄⁺.

In specific embodiments, the modified sophorolipid comprises taurine, which is derived from a truncated sophorolipid (sophorolipid with a truncated fatty acid chain) covalently linked to taurine through an amide bond. In a specific embodiment, the modified sophorolipid has a structure of:

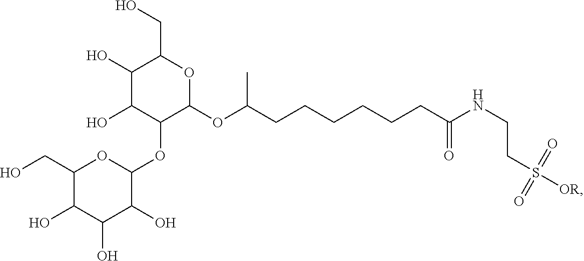

wherein R is hydrogen or a cation selected from, for example, Na⁺, K⁺, Li⁺, Cs⁺, Rb⁺, Ca²⁺, Mg²⁺ and NH₄⁺.

In specific embodiments, the modified sophorolipid comprises taurine, which is derived from a truncated sophorolipid (sophorolipid with a truncated fatty acid chain) covalently linked to taurine through an amine. In a specific embodiment, the modified sophorolipid has a structure of:

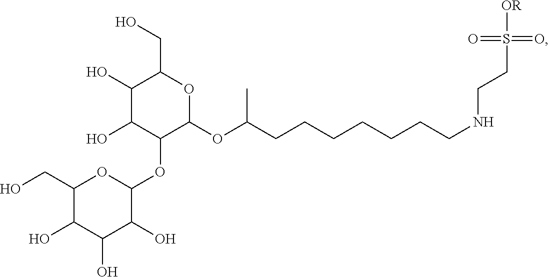

wherein R is hydrogen or a cation selected from, for example, Na⁺, K⁺, Li⁺, Cs⁺, Rb⁺, Ca²⁺, Mg²⁺ and NH₄⁺.

### Selected Definitions

As used herein, the term “foam” refers to a dispersion of gas bubbles in or on a liquid, in a gel or in a semisolid. The gas bubbles may be dispersed throughout the liquid phase in a heterogeneous or homogeneous manner. Illustrative examples of foams include gases such as air, nitrogen, oxygen, helium or hydrogen entrapped in a liquid such as water or an oil. A foam may be transient, unstable or stable.

As used herein, “surfactant” refers to a substance or compound that reduces surface tension when dissolved in water or water solutions, or that reduces interfacial tension between two liquids, or between a liquid and a solid. The term “surfactant” thus includes cationic, anionic, nonionic, zwitterionic, amphoteric agents and/or combinations thereof. By “biosurfactant” is meant a surface active agent produced by a living organism, and/or produced using naturally-derived substrates. “surfactant” and “biosurfactant” also pertain to substances that cause foaming.

As used herein, the term “alkyl” refers to straight chain or branched hydrocarbon groups. Suitable alkyl groups include, but are not limited to methyl, ethyl, propyl, isopropyl, butyl, isobutyl, pentyl, hexyl, heptyl, octyl, nonyl, decyl, dodecyl, tetradecyl, hexadecyl and octadecyl. The term alkyl may be prefixed by a specified number of carbon atoms to indicate the number of carbon atoms or a range of numbers of carbon atoms that may be present in the alkyl group such as C1-C10 alkyl, C1-C20 alkyl, and C10-C20 alkyl. For example, C1-C3 alkyl refers to methyl, ethyl, propyl and isopropyl.

As used herein, the term “alkenyl” refers to straight chain or branched hydrocarbon groups containing at least one double bond. Suitable alkenyl groups include, but are not limited to vinyl, propenyl, 1-butenyl, 2-butenyl, 3-butenyl, 3-methyl-2-butenyl, 1-pentenyl, 2-pentenyl, 3-pentenyl, 4-pentenyl, 3-methyl-2-pentenyl, 4-methyl-3-pentenyl, 2,4-pentadiene, 1-hexenyl, 2-hexenyl, 3-hexenyl, 4-hexenyl, 5-hexenyl, 3-methyl-2-hexenyl, 4-methyl-3-hexenyl and 5-methyl-4-hexenyl. The term alkenyl may be prefixed by a specified number of carbon atoms to indicate the number of carbon atoms or a range of numbers of carbon atoms that may be present in the alkenyl group such as C1-C10 alkenyl, C1-C20 alkenyl, and C10-C20 alkenyl.

As used herein, “alkenylene” refers to a straight or branched, divalent carbon chain having one or more carbon-carbon double bonds (—CR=CR′—, wherein R and R′ are each independently hydrogen or further substituents). “Alkenylene” refers to an unsaturated, branched or straight chain radical of 2-18 carbon atoms, and having two monovalent radical centers derived by the removal of two hydrogen atoms from the same or two different carbon atoms of a parent alkene. Examples of alkenylene include —CH═CH—, —CH2CH═CHCH2-, —CH2CH2CH═CHCH2CH2-, —CH2CH═CHCH2CH2CH2-, —CH2CH═CHCH2CH2CH═CHCH2-, —CH2CH2CH2CH═CHCH2CH2CH2-, —CH2CH2CH2CH2CH═CHCH2CH2CH2CH2CH2CH2-, —CH2CH2CH2CH2CH═CHCH2CH2CH2CH═CHCH2CH2CH2-, and the like. The term alkenylene may be prefixed by a specified number of carbon atoms to indicate the number of carbon atoms or a range of numbers of carbon atoms that may be present in the alkenylene group such as Cl—Cl0 alkenylene, C1-C20 alkenylene, and C5-C15 alkenylene.

As used herein, “alkylene” refers to a bivalent saturated, straight or branched, aliphatic radical (such as ethylene) from an alkane by removal of two hydrogen atoms from different carbon atoms. Examples of alkylene include, but are not limited to, methylene (—CH2-) 1,2-ethyl (—CH2CH2-), 1,3-propyl (—CH2CH2CH2-), 1,4-butyl (—CH2CH2CH2CH2-), —CH(CH3)CH2-, —CH2CH2CH2CH2CH2CH2-, —CH2CH2CH2CH2CH2CH2CH2CH2-, and the like. The term alkylene may be prefixed by a specified number of carbon atoms to indicate the number of carbon atoms or a range of numbers of carbon atoms that may be present in the alkylene group such as C1-C10 alkylene, C1-C20 alkylene, and C5-C15 alkylene.

As used herein, an “isolated” or “purified” nucleic acid molecule, polynucleotide, polypeptide, protein or organic compound, such as a small molecule, is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of other molecules, or the amino acids that flank it, in its naturally-occurring state. An “isolated” strain means that the strain is removed from the environment in which it exists in nature. Thus, the isolated strain may exist as, for example, a biologically pure culture, or as spores (or other forms of the strain).

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein “fermentation” refers to growth or cultivation of cells under controlled conditions. The growth could be aerobic or anaerobic. Unless the context requires otherwise, the phrase is intended to encompass both the growth phase and product biosynthesis phase of the process.

As used herein, a “broth,” “culture broth,” or “fermentation broth” refers to a culture medium comprising at least nutrients. If the broth is referred to after a fermentation process, the broth may comprise microbial growth byproducts and/or microbial cells as well.

The microbe growth vessel used according to the subject invention can be any fermenter or cultivation reactor for industrial use. As used herein, the term “reactor,” “bioreactor,” “fermentation reactor” or “fermentation vessel” includes a fermentation device consisting of one or more vessels and/or towers or piping arrangements. Examples of such reactor includes, but are not limited to, the Continuous Stirred Tank Reactor (CSTR), Immobilized Cell Reactor (ICR), Trickle Bed Reactor (TBR), Bubble Colunm, Gas Lift Fermenter, Static Mixer, or other vessel or other device suitable for gas-liquid contact. In some embodiments, the bioreactor may comprise a first growth reactor and a second fermentation reactor. As such, when referring to the addition of substrate to the bioreactor or fermentation reaction, it should be understood to include addition to either or both of these reactors where appropriate.

As used herein, “reduces” means a negative alteration, and “increases” means a positive alteration, wherein the alteration is at least 0.001%, 0.01%, 0.1%, 1%, 5%, 10%, 15%, 20%, 25%, 30%, 35%, 45%, 50%, 55%, 60%, 65%, 70%, 75%, 80%, 85%, 90%, 95%, 99%, or 100%, inclusive of all values therebetween.

The term “subject” or “patient,” as used herein, describes an organism, including mammals such as primates. Mammalian species that can benefit from the disclosed methods of treatment include, but are not limited to, apes, chimpanzees, orangutans, humans, and monkeys; domesticated animals such as dogs, cats; live stocks such as horses, cattle, pigs, sheep, goats, and chickens; and other animals such as mice, rats, guinea pigs, and hamsters.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof. All references cited herein are hereby incorporated by reference.

### Production of Sophorolipids

The subject invention provides materials and methods for producing and modifying sophorolipids. Advantageously, the subject invention is suitable for industrial scale production of purified SLP and uses safe and environmentally-friendly materials and processes.

Sophorolipids are generally obtained from fermentations by microorganisms that use as carbon sources pure fatty acids, fatty acid mixtures, pure fatty acid esters, mixtures of fatty acid esters, triglycerides along with carbohydrate sources such as corn syrup, dextrins and glucose using a fermentation process comprising a wild-type or engineered microorganism. The production of sophorolipids with the use of renewable substrates and different microbial species, as well as the variation in culture parameters (incubation time, stirring speed, pH of the medium and added nutrients), allow for the acquisition of compounds with distinct structural and physical properties. This makes it possible to produce a wide variety of compounds that can elicit different physical, chemical, biochemical, and biophysical properties.

In preferred embodiments, the subject methods initially comprise producing standardized sophorolipid molecules for producing modified and/or purified sophorolipids. In certain embodiments, this entails cultivating a sophorolipid-producing yeast in a submerged fermentation reactor comprising a tailored oleochemical feedstock to produce a yeast culture product, said yeast culture product comprising fermentation broth, yeast cells and sophorolipids having a mixture of two or more molecular structures.

The mixture of molecular structures can comprise, for example, lactonic sophorolipids, linear sophorolipids, de-acetylated sophorolipids, mono-acetylated sophorolipids, di-acetylated sophorolipids, esterified sophorolipids, sophorolipids with varying hydrophobic chain lengths, sophorolipids with fatty acid-amino acid complexes attached, and others, including those that are and/or are not described within in this disclosure.

In certain embodiments, the distribution of the mixture of sophorolipids molecules can be altered by adjusting fermentation parameters, such as, for example, feedstock, fermentation time, and dissolved oxygen levels.

The sophorolipids according to the present invention can be derived via a fermentation process from a recombinant organism or by a strain that naturally produces sophorolipids. Non-limiting examples of sophorolipid-producing organisms include Candida bombicola, Candida apicola, Candida bogoriensis, Yarrowia lipolytica, Starmerella bombicola, Starmerella clade, Rhodotorula bogoriensis, Wickerhamiella domericqiae, and Wickerhamomyces anomalus. Some recombinant sophorolipid-producing microbes have been reported to allow control of sophorolipid structure. As a non-limiting example, certain recombinant S. bombicola strains may be utilized to produce either solely lactonic or solely acidic sophorolipids. In addition, a recombinant Candida bombicola strain with an acetyltransferase gene knockout can be used to produce sophorolipids without acetylation.

In preferred embodiments, the microorganism is a yeast or fungus. Examples of yeast and fungus species suitable for use according to the current invention, include, but are not limited to, Acaulospora, Aspergillus, Aureobasidium (e.g., A. pullulans), Blakeslea, Candida (e.g., C. albicans, C. apicola), Cryptococcus, Debaryomyces (e.g., D. hansenii), Entomophthora, Fusarium, Hanseniaspora (e.g., H. uvarum), Hansenula, Issatchenkia, Kluyveromyces, Mortierella, Mucor (e.g., M. piriformis), Meyerozyma (e.g., M. guilliermondii), Penicillium, Phythium, Phycomyces, Pichia (e.g., P. anomala, P. guilliermondii, P. occidentalis, P. kudriavzevii), Pseudozyma (e.g., P. aphidis), Rhizopus, Saccharomyces (S. cerevisiae, S. boulardii sequela, S. torula), Starmerella (e.g., S. bombicola), Torulopsis, Thraustochytrium, Trichoderma (e.g., T. reesei, T. harzianum, T. virens), Ustilago (e.g., U. maydis), Wickerhamomyces (e.g., W. anomalus), Williopsis, and Zygosaccharomyces (e.g., Z. bailii).

In preferred embodiments, the microorganisms are selected from, for example, Starmerella spp. yeasta and/or Candida spp. yeasta, e.g., Starmerella (Candida) bombicola, Candida apicola, Candida batistae, Candida floricola, Candida riodocensis, Candida stellate and Candida kuoi. In a specific embodiment, the microorganism is Starmerella bombicola, e.g., strain ATCC 22214.

In one embodiment, the fermentation reactor may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken from the vessel for enumeration, purity measurements, SLP concentration, and/or visible oil level monitoring. For example, in one embodiment, sampling can occur every 24 hours.

The microbial inoculant according to the subject methods preferably comprises cells and/or propagules of the desired microorganism, which can be prepared using any known fermentation method. The inoculant can be pre-mixed with water and/or a liquid growth medium, if desired.

The microorganisms utilized according to the subject invention may be natural, or genetically modified microorganisms. For example, the microorganisms may be transformed with specific genes to exhibit specific characteristics. The microorganisms may also be mutants of a desired strain.

In one embodiment, a single type of microorganism is grown in a reactor system. In alternative embodiments, multiple microorganisms, which can be grown together without deleterious effects on growth or the resulting product, can be grown in a single reactor system. There may be, for example, 2 to 3 or more different microorganisms grown in a single reactor at the same time. In some embodiments, more than one microorganism grows symbiotically in the reactor.

In certain embodiments, the cultivation may be supplemented with a carbon source. The carbon source can be a carbohydrate, such as glucose, dextrose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as canola oil, soybean oil, rice bran oil, olive oil, corn oil, sunflower oil, sesame oil, and/or linseed oil; powdered molasses, etc. These carbon sources may be used independently or in a combination of two or more.

In certain embodiments, the cultivation may be supplemented with high-oleic acid and/or exclusively-oleic acid oleochemical feedstock, which results in a yeast culture product comprising a narrower diversity of sophorolipid molecular structures than with feedstocks containing sources of other fatty acids, wherein the principal sophorolipid molecules produced contain an 18C carbon chain and a single unsaturated bond at the ninth carbon.

In one embodiment, the liquid growth medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid growth medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included.

The method of cultivation can further provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid.

In one embodiment, the microorganisms can be grown on a solid or semi-solid substrate, such as, for example, corn, wheat, soybean, chickpeas, beans, oatmeal, pasta, rice, and/or flours or meals of any of these or other similar substances.

In one embodiment, inorganic salts may also be included. Usable inorganic salts can be potassium dihydrogen phosphate, dipotassium hydrogen phosphate, disodium hydrogen phosphate, magnesium sulfate, magnesium chloride, iron sulfate, iron chloride, manganese sulfate, manganese chloride, zinc sulfate, lead chloride, copper sulfate, calcium chloride, calcium carbonate, sodium chloride, and/or sodium carbonate. These inorganic salts may be used independently or in a combination of two or more.

In some embodiments, when, for example, the microbes used to inoculate the substrate are in spore form (e.g., bacterial endospores), germination enhancers can be added to the substrate. Examples of germination enhancers according to the present invention include, but are not limited to, L-alanine, manganese, L-valine, and L-asparagine or any other known germination enhancer.

The pH of the culture should be suitable for the microorganism of interest. Buffers, and pH regulators, such as carbonates and phosphates, may be used to stabilize pH near a preferred value. In certain embodiments, a base solution is used to adjust the pH of the culture to a favorable level, for example, a 15% to 30%, or a 20% to 25% NaOH solution. The base solution can be included in the growth medium and/or it can be fed into the fermentation reactor during cultivation to adjust the pH as needed. When metal ions are present in high concentrations, use of a chelating agent in the liquid medium may be necessary.

The method and equipment for cultivation of microorganisms and production of the microbial by-products can be performed in a batch, quasi-continuous, or continuous processes.

In one embodiment, the method of cultivation is carried out at about 5° to about 100° C., about 15° to about 60° C., about 20° to about 45° C., about 22° to about 35° C., or about 24° to about 28° C. In one embodiment, the cultivation may be carried out continuously at a constant temperature. In another embodiment, the cultivation may be subject to changing temperatures.

According to the subject methods, the microorganisms can be cultivated in the fermentation system for a time period sufficient to achieve a desired effect, e.g., production of a desired amount of cell biomass or a desired amount of sophorolipids.

In certain embodiments, fermentation of the yeast culture occurs for about 40 to 150 hours, or about 48 to 140 hours, or about 72 to 130 hours or about 96 to 120 hours. In certain specific embodiments, fermentation time ranges from 48 to 72 hours, or from 96 to 120 hours.

In some embodiments, the sophorolipids produced by microorganisms of interest may be retained in the microorganisms or secreted into their growth medium. The sophorolipid content can be, for example, at least 20%, 30%, 40%, 50%, 60%, 70%, 80%, or 90%.

The growth medium may contain compounds that stabilize the activity of the sophorolipids. The sophorolipids can be purified, or the sophorolipids can be used in crude form, meaning they are not separated from the fermentation broth in which they were produced.

In certain embodiments, the sophorolipid is isolated and/or purified from the growth medium resulting from fermentation of a biosurfactant-producing microorganism. Isolation and purification can be easily achieved using standard methods or techniques described in the literature. The sophorolipid can be further concentrated, if desired.

### Modification of Sophorolipids

In some embodiments, the modified sophorolipids according to the present invention are obtained by chemically modifying sophorolipids that have been obtained from the product of fermentation by sophorolipid-producing microbes. The sophorolipids to be chemically modified may be obtained by fermentation methods and processes as described above and/or any other methods known in the art. Any suitable techniques and chemical reactions known in the art may be used to modify sophorolipids. For example, amide coupling reactions known in the art may be utilized to form an amide bond between taurine and the carboxyl group of sophorolipids.

Chemical modifications can also be made, for example, to alter the degree of unsaturation on the fatty acid chain. Commonly known hydrogenation or dehydrogenation reactions, or addition or elimination reactions may be used.

Sophorolipids containing an unsaturated bond at a specific position allows for site-directed functionalization of the SLP molecule. In certain embodiments, the linear SLP are ozonated with ozone gas. During ozonolysis of the linear SLP, the olefin moiety of the SLP molecule is converted to an ozonide, a reactive 5-membered ring. In preferred embodiments, the sophorolipid containing the ozonide is reduced to afford an aldehyde handle. In a specific embodiment, the reducing agent is triphenyl phosphine used in equimolar concentrations to the SLP-ozonide.

In preferred embodiments, the linear sophorolipid aldehyde is further reduced in the presence of taurine and a reducing agent to afford the modified sophorolipid where taurine is covalently added to sophorolipid through an amine formation. In certain embodiments, the reducing agent is sodium cyanoborohydride, sodium triacetoxyborohydride or sodium borohydride.

In some embodiments, the linear sophorolipid aldehyde is extracted with ethyl acetate from the aqueous mixture and concentrated and dried under reduced pressure (e.g., about 200 to 250 mbar, or about 240 mbar) at a temperature of about 35 to 45° C. The dried crude linear sophorolipid aldehyde can then be dissolved in a reaction medium comprising tetrahydrofuran (THF) and water. The percentage of water used as the reaction medium preferably does not exceed 50% water, and typically is between 0 to 25%.

In certain embodiment, the sophorolipid is undergo an amination reaction, where taurine is introduced to the linear sophorolipid aldehyde mixture along with a reducing agent and optionally, a weak organic acid, preferably acetic acid, although other organic acids may be used (e.g., formic acid, trifluoracetic acid).

In some embodiments, methods are provided for producing a modified sophorolipid composition, the methods comprising providing one or more sophorolipid molecules; functionalizing the sophorolipid molecule(s) to covalently linked to taurine through an amide bond or amine bond to form modified sophorolipid molecule(s); and, optionally, mixing the modified sophorolipid molecule(s) with a liquid, with other sophorolipid molecules, and/or with additional components specific to the type of product being produced.

One embodiment is directed to a method for producing the modified sophorolipids comprising: obtaining the linear sophorolipid molecules; and functionalizing the linear sophorolipid molecules by converting all, or a majority, of the linear SLP molecules to a modified sophorolipid molecules.

### Compositions

In one embodiment, the subject invention provides compositions comprising the modified sophorolipids of the subject invention. In certain embodiments, the modified sophorolipids produced according to the subject methods can be used as active ingredients in personal care products.

In some embodiment, the composition comprises, for example, at least, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50% (wt %) of the modified sophorolipids. In certain embodiments, the composition comprises the modified sophorolipids at 0.1 to 50% by weight, for example, 0.1 to 45%, 0.1 to 40%, 0.1 to 35%, 0.1 to 30%, 0.1 to 25%, 0.1 to 20%, 0.1 to 20%, 0.1 to 15%, 0.1 to 10%, 0.1 to 9.0%, 0.1 to 8.0%, 0.1 to 7.0%, 0.1 to 6.0%, 0.1 to 5.0%, 0.1 to 4.0%, 0.1 to 3.0%, 0.1 to 2.0%, 1.0 to 9.0%, 1.0 to 5.0%, 1.0 to 3.0%, 3.0 to 10%, 3.0 to 7.0%, 5.0 to 10%, 5.0 to 9.0%, 6.0 to 10%, 7.0 to 10%, 8.0 to 10%, 5 to 40%, 10 to 40%, 10 to 30%, or 10 to 20%.

Optionally, the composition can further comprise one or more other components, including, for example, carriers (e.g., water), other biosurfactants, other surfactants (e.g., polyalkyglucosides such as capryl glucoside and lauryl glucoside), hydrophilic and/or hydrophobic syndetics, sequestrants, builders (e.g., potassium carbonate, sodium hydroxide, glycerin, citric acid, lactic acid), solvents (e.g., water, ethanol, methanol, isopropanol), organic and/or inorganic acids (e.g., lactic acid, citric acid, boric acid), essential oils, botanical extracts, cross-linking agents, chelators (e.g., potassium citrate), fatty acids, alcohols, pH adjusting agents, reducing agents, calcium salts, carbonate salts, buffers, enzymes, dyes, colorants, fragrances, preservatives (e.g., octylisothiazolinone, methylisothiazolinone), terpenes (e.g., d-limonene), sesquiterpenoids, terpenoids, emulsifiers, demulsifiers, foaming agents, defoamers, bleaching agents, polymers, thickeners and/or viscosifiers (e.g., xanthan gum, guar gum).

In some embodiments, the composition comprises additional biosurfactants. Additional biosurfactants according to the subject invention can include, for example, glycolipids, lipopeptides, flavolipids, phospholipids, fatty acid esters, and high-molecular-weight biopolymers such as lipoproteins, lipopolysaccharide-protein complexes, and/or polysaccharide-protein-fatty acid complexes.

In one embodiment, the additional biosurfactant is a glycolipid, such as, for example, rhamnolipids (RLP), cellobiose lipids, trehalose lipids and/or mannosylerythritol lipids (MEL). In one embodiment, the biosurfactant is a lipopeptide, such as, for example, surfactin, iturin, fengycin, arthrofactin, amphisin, viscosin, lichenysin, paenibacterin, polymyxin and/or battacin, In one embodiment, the biosurfactant is another type of amphiphilic molecule, such as, for example, esterified fatty acids, saponins, cardiolipins, pullulan, emulsan, lipomanan, alasan, and/or liposan.

The composition may further include additional active or inactive ingredients, suitable for the mode of administration and intended purpose, provided that such addition does not adversely interfere with the functions of the modified sophorolipids.

In certain embodiments, acceptable carriers may be included in the composition of the present invention, depending on the form of the composition and/or mode of administration. Pharmaceutically or cosmetically acceptable carriers include, but are not limited to, pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid, or tale. If desired or suitable, a coating material may also be used such as glyceryl monostearate or glyceryl distearate, for example, to delay absorption in the gastrointestinal tract if appropriate and the pharmaceutical composition is in the form of a solid form.

In certain embodiments, the subject invention provides topical skin care compositions comprising one or more of the modified sophorolipids. In some embodiments, the topical composition can further comprise a dermatologically acceptable carrier, and one or more active or inactive cosmetic ingredients, such as, e.g., vitamins, moisturizers, dyes, fragrances, sunscreens, exfoliants, essential oils, botanical extracts, and so on.

The dermatologically acceptable carrier may include, for example, water; saline; physiological saline; ointments; creams; oil-water emulsions; water-in-oil emulsions; silicone-in-water emulsions; water-in-silicone emulsions; wax-in-water emulsions; water-oil-water triple emulsions; microemulsions; gels; vegetable oils; mineral oils; ester oils such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane and isohexadecane; silicone oils such as cyclomethicone, dimethicone, dimethicone cross-polymer, polysiloxanes and their derivatives, preferably organo-modified derivatives including PDMS, dimethicone copolyol, dimethiconols, and amodimethiconols; hydrocarbon oils such as mineral oil, petrolatum, isoeicosane and polyolefins, e.g., (hydrogenated) polyisobutene; polyols such as propylene glycol, glycerin, butylene glycol, pentylene glycol, hexylene glycol, caprylyl glycol; waxes such as beeswax, carnauba, ozokerite, microcrystalline wax, polyethylene wax, and botanical waxes; or any combinations or mixtures of the foregoing. Aqueous carriers may include one or more solvents miscible with water, including lower alcohols, such as ethanol, isopropanol, and the like. The carrier may comprise from about 1% to about 99% by weight of the composition, from 10% to about 85%, from 25% to 75%, or from 50% to about 65%.

In some embodiments, the composition is in solid form and suitable for reconstitution in a solvent. In other embodiments, the composition further comprises a solvent. Suitable solvents may be aqueous or non-aqueous and are preferably suitable for foam formation. In particular embodiments, the solvent is an aqueous solvent. Examples of suitable solvents include water, buffer, acetonitrile, water and alcohol mixtures such as aqueous methanol, aqueous ethanol and aqueous isopropanol. In some embodiments, the foam may optionally comprise an emulsion, which is a dispersion of oil in water.

Non-biological surfactants can also be added to the formulation. Examples of surfactants include, but are not limited to, alkyl sulfates, alkyl ether sulfates (e.g., sodium/ammonium lauryl sulfates and sodium/ammonium laureth sulfates), amphoterics (e.g., amphoacetates and amphopropionates), sulfosuccinates, alkyl polyglucosides, betaines (e.g., cocamidopropyl betaine), sultaines, sacrosinates, isethionates, taurates, ethoxylated sorbitan esters, alkanolamides and amino acid-based surfactants.

Viscosity modifiers can also be added to the compositions, including, for example, cocamide DEA, oleamide DEA, sodium chloride, cellulosic polymers, polyacrylates, ethoxylated esters, alcohol, glycols, xylene sulfonates, polysorbate 20, alkanolamides, and cellulose derivatives (e.g., hydroxypropyl methylcellulose and hydroxyethyl cellulose).

Polymers can also be added, including, for example, xanthan gum, guar gum, polyquaternium-10, PEG-120 methyl glucose dioleate, PEG-150 distearate, PEG-150 polyglyceryl-2 tristearate and PEG-150 pentaerythrityl tetrastearate.

A sunscreen or combination of sunscreens may be included to protect the skin from both UVA and UVB rays. Among the sunscreens that can be employed in the present compositions are avobenzone, cinnamic acid derivatives (such as octylmethoxy cinnamate), octyl salicylate, oxybenzone, octocrylene, titanium dioxide, zinc oxide, or any mixtures thereof. The sunscreen may be present from about 1 wt % to about 30 wt % of the total weight of the composition.

In one embodiment, the composition may include additional skin actives, including but not limited to, keratolytic agents, desquamating agents, keratinocyte proliferation enhancers, collagenase inhibitors, elastase inhibitors, depigmenting agents, anti-inflammatory agents, steroids, anti-acne agents, antioxidants (e.g., ascorbic acid), advanced glycation end-product (AGE) inhibitors, exfoliating agents (e.g., glycolic acid, 3,6,9-trioxaundecanedioic acid, etc.), estrogen synthetase stimulating compounds (e.g., caffeine and derivatives), compounds capable of inhibiting 5 alpha-reductase activity (e.g., linolenic acid, linoleic acid, finasteride, and mixtures thereof), barrier function enhancing agents (e.g., ceramides, glycerides, cholesterol and its esters, alpha-hydroxy and omega-hydroxy fatty acids and esters thereof), and retinoids.

The composition may optionally comprise additives, adjuvants, or other components of topical compositions known to those skilled in the art including, but not limited to: skin penetration enhancers; emollients (e.g., isopropyl myristate, petrolatum, volatile or non-volatile silicones oils, such as methicone and dimethicone, ester oils, mineral oils, and fatty acid esters); humectants (e.g., glycerin, hexylene glycol, caprylyl glycol); skin plumpers (e.g., palmitoyl oligopeptide, collagen, collagen and/or glycosaminoglycan (GAG) enhancing agents); anti-inflammatory agents (e.g., Aloe vera, bioflavonoids, diclofenac, salicylic acid); chelating agents (e.g., EDTA or a salt thereof, such as disodium EDTA); vitamins (e.g., tocopherol and ascorbic acid); vitamin derivatives (e.g., ascorbyl monopalmitate, tocopheryl acetate, Vitamin E palmitate); thickeners (e.g., hydroxyalkyl cellulose, carboxymethylcellulose, carbombers, and vegetable gums, such as xanthan gum); gelling agents (e.g., ester-terminated polyester amides); structuring agents; proteins (e.g., lactoferrin); immune modulators (e.g., corticosteroids and non-steroidal immune modulators). Further examples include, but are not limited to: stabilizers; fragrances; film formers; insect repellents; skin cooling compounds; skin protectants; lubricants; preservatives; pearls; chromalites; micas; conditioners; anti-allergenics; pH adjusters; and antimicrobials.

In some embodiments, the topical composition can further comprise additional adjuvants and additives commonly included in skin care compositions, such as, for example, organic solvents, stabilizers, silicones, thickeners, softeners, sunscreens, moisturizers, fragrances or others described herein. The amounts of each ingredient, whether active or inactive, are those conventionally used in the cosmetic field to achieve their intended purpose, and typically range from about 0.0001% to about 25%, or from about 0.001% to about 20% of the composition, although the amounts may fall outside of these ranges. The nature of these ingredients and their amounts must be compatible with the production and function of the compositions of the disclosure.

In certain embodiments, the composition may optionally include a skin conditioner such as an emollient, humectant, occlusive agent, or other moisturizer to provide moisturization, skin softening, skin barrier maintenance, anti-irritation, or other skin health benefits. Some non-limiting examples of emollients include alkyl benzoate, myristyl myristate, cetyl myristate, glyceryl dioleate, methyl laurate, PPG-9 laurate, lauryl lacylate allantoin, octyl palmitate, lanolin, propylene glycol, butylenes glycol, ethylene glycol monobutyl ether, glycerine, fatty acids, natural oils such as almond, mineral, canola, sesame, soybean, wheat germ, corn, peanut and olive oil, isopropyl myristate, myristyl alcohol, aloe vera, hydrolyzed silk protein, Vitamin E, stearyl alcohol, isopropyl palmitate, sorbitol, amino acid complexes, hydrolyzed plant proteins, and polyethylene glycol. Some non-limiting examples of humectants include hydroxyethyl urea, agarose, urea, sodium PCA, arginine PCA, fructose, glucose, glutamic acid, glycerine, honey, lactose, maltose, polyethylene glycol, sorbitol and mixtures thereof. Some non-limiting examples of occlusive agents include petrolatum, shea butter, avocado oil, balm mint oil, cod liver oil, mineral oil, trimyristin, stearyl stearate, synthetic wax, or mixtures thereof. Some non-limiting examples of other moisturizers include ethyl hexylglycerin, cholesterol, cystine, hyaluronic acid, keratin, lecithin, egg yolk, glycine, PPG-12, polyquaternium polymers such as polyquaternium-11, benentrimonium chloride, dihydroxypropyl PEG-5 linoleammonium chloride, glycerol oleate, PEG-7 glyceryl cocoate, cocoglucoside, PEG-200 hydrogenated glyceryl palmate, panthenol, retinol, salicylic acid, vegetable oil, methyl gluceth-10, methyl gluceth-20, ethoxylated derivatives of skin conditioners such as glycereth-26 and ethoxylated shea butter, and mixtures thereof. Finally, some non-limiting examples of anti-irritants include bisabolol and panthenol.

A person skilled in the art will recognize the different strengths of different skin conditioners and formulate accordingly. In some embodiments, the skin conditioner is preferably present in the composition in an amount from about 0.01 to about 20 wt. %, from about 0.05 to about 15 wt. %, and from about 0.1 to about 10 wt. %.

In some embodiments, the composition may optionally include molecules that enhance the “feel” of the composition on a user's skin or hands. For example, it may be undesirable for a composition to have a scaly or gritty texture when applied to a user's skin or after the multiple applications of the composition. Some non-limiting examples include silicone copolymers such as amodimethicone, cyclomethicone, bis-PEG/PPG-20/20 dimethicone, and stearoxytrimethylsilane, naturally occurring or synthetic fatty acid esters or ethers, and polyalkylene glycols. If included, it is preferably present in the composition in an amount from about 0.001 to about 5 wt. %, from about 0.01 to about 3 wt. %, and from about 0.1 to about 2 wt. %.

In one embodiment, the composition may include anti-aging components, including, but not limited to, botanicals (e.g., Butea frondosa extract); phytol; phytonic acid; phospholipids; silicones; petrolatum; triglycerides; omega fatty acids; retinoids; hydroxy acids (including alpha-hydroxy acids and beta-hydroxy acids), salicylic acid and alkyl salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof.)

Exemplary retinoids include, without limitation, retinoic acid (e.g., all-trans, or 9-cis, or 13-cis), and derivatives thereof, retinaldehyde, retinol (Vitamin A) and esters thereof, such as retinyl palmitate, retinyl acetate and retinyl propionate, and salts thereof. When present, the retinoids will typically be included in amounts from about 0.0001% to about 5% by weight, more typically from about 0.01% to about 2.5% by weight, or from about 0.1% to about 1.0% by weight.

In one embodiment, the composition may include an exfoliating agent. Suitable exfoliating agents include, for example, alpha-hydroxy acids, beta-hydroxy acids, oxa-acids, oxadiacids, and their derivatives, such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. One exemplary exfoliating agent is glycolic acid. When present, the exfoliating agent may comprise from about 0.001% to about 20% by weight of the composition.

In one embodiment, the composition may comprise one or more antioxidants. Suitable antioxidants include, for example, compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives, including tocopheryl acetate; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Antioxidants may comprise, individually or collectively, from about 0.001% to about 10% (w/w), or from about 0.01% to about 5% (w/w) of the total weight of the composition.

In some embodiment, the composition may optionally include a fragrance. Examples of possible fragrances include natural oils or naturally derived materials, and synthetic fragrances such as hydrocarbons, alcohols, aldehydes, ketones, esters, lactones, ethers, nitriles, and polyfunctionals. Non-limiting examples of natural oils include the following: basil (Ocimum basilicum) oil, bay (Pimento acris) oil, bee balm (Monarda didyma) oil, bergamot (Citrus aurantium bergamia) oil, cardamom (Elettaria cardamomum) oil, cedarwood (Cedrus atlantica) oil, chamomile (Anthemis nobilis) oil, cinnamon (Cinnamomum cassia) oil, citronella (Cymbopogon nardus) oil, clary (Salvia sclarea) oil, clove (Eugenia caryophyllus) oil, cloveleaf (Eufenia caryophyllus) oil, Cyperus esculentus oil, cypress (Cupressus sempervirens) oil, Eucalyptus citriodora oil, geranium maculatum oil, ginger (Zingiber officinale) oil, grapefruit (Citrus grandis) oil, hazel (Corylus avellana) nut oil, jasmine (Jasminum officinale) oil, Juniperus communis oil, Juniperus oxycedrus tar, Juniperus virginiana oil, kiwi (Actinidia chinensis) water, lavandin (Lavandula hybrida) oil, lavender (Lavandula angustifolia) oil, lavender (Lavandula angustifolia) water, lemon (Citrus medica limonum) oil, lemongrass (Cymbopogon schoenanthus) oil, lime (Citrus aurantifolia) oil, linden (Tilia cordata) oil, linden (Tilia cordata) water, mandarin orange (Citrus nobilis) oil, nutmeg (Myristica fragrans) oil, orange (Citrus aurantium dulcis) flower oil, orange (Citrus aurantium dulcis) oil, orange (Citrus aurantium dulcis) water, patchouli (Pogostemon cablin) oil, peppermint (Menthe piperita) oil, peppermint (Menthe peperita) water, rosemary (Rosmarinus officinalis) oil, rose oil, rose (Rosa damascena) extract, rose (Rosa multiflora) extract, rosewood (Aniba rosaeodora) extract, sage (Salvia officinalis) oil, sandalwood (Santalum album) oil, spearmint (Menthe viridis) oil, tea tree (Melaleuca alternifolia) oil, and ylang ylang (Cananga odorata) oil. Some non-limiting examples of synthetic hydrocarbon fragrances include caryophyllene, β-famesene, limonene, α-pinene, and β-pinene. Some non-limiting examples of synthetic alcohol fragrances include bacdanol, citronellol, linalool, phenethyl alcohol, and α-terpineol (RaH). Some non-limiting examples of synthetic aldehyde fragrances include 2-methyl undecanal, citral, hexyl cinnamic aldehyde, isocycolcitral, lilial, and 10-undecenal. Some non-limiting examples of synthetic ketone fragrances include cashmeran, α-ionone, isocyclemone E, koavone, muscone, and tonalide. Some non-limiting examples of synethetic ester fragrances include benzyl acetate, 4-t-butylcyclohexyl acetate (cis and trans), cedryl acetate, cyclacet, isobornyl acetate, and α-terpinyl acetate (R=acetyl). Some non-limiting examples of synthetic lactone fragrances include coumarin, jasmine lactone, muskalactone, and peach aldehyde. Some non-limiting examples of synthetic ether fragrances include ambroxan, anther, and galaxolide. Some non-limiting examples of synthetic nitrile fragrances include cinnamonitrile and gernonitrile. Finally, some non-limiting examples of synthetic polyfunctional fragrances include amyl salicylate, isoeugenol, hedione, heliotropine, lyral, and vanillin.

The composition may include a mixture of fragrances including a mixture of natural and synthetic fragrances. The fragrance can be present in a composition in an amount up to about 5 wt. %, preferably from about 0.01 to about 3 wt. %, from about 0.05 to about 1 wt. %, and from about 0.1 to about 0.2 wt. %.

In some embodiments, the composition may optionally include a preservative. Generally, preservatives fall into specific classes including phenolics, halogen compounds, quaternary ammonium compounds, metal derivatives, amines, alkanolamines, nitro derivatives, biguanides, analides, organosulfur and sulfur-nitrogen compounds, alkyl parabens, and miscellaneous compounds. Some non-limiting examples of phenolic antimicrobial agents include pentachlorophenol, orthophenylphenol, chloroxylenol, p-chloro-m-cresol, p-chlorophenol, chlorothymol, m-cresol, o-cresol, p-cresol, isopropyl cresols, mixed cresols, phenoxyethanol, phenoxyethylparaben, phenoxyisopropanol, phenyl paraben, resorcinol, and derivatives thereof. Some non-limiting examples of halogen compounds include trichlorohydroxy diphenyl ether (Triclosan), sodium trichloroisocyanurate, sodium dichloroisocyanurate, iodine-poly(vinylpyrolidin-onen) complexes, and bromine compounds such as 2-bromo-2-nitropropane-1,3-diol, and derivatives thereof. Some non-limiting examples of quaternary ammonium compounds include benzalkonium chloride, benzethonium chloride, behentrimonium chloride, cetrimonium chloride, and derivatives thereof. Some non-limiting examples of amines and nitro containing compounds include hexahydro-1,3,5-tris(2-hydroxyethyl)-s-triazine, dithiocarbamates such as sodium dimethyldithiocarbamate, and derivatives thereof. Some non-limiting examples of biguanides include polyaminopropyl biguanide and chlorhexidine gluconate. Some non-limiting examples of alkyl parabens include methyl, ethyl, propyl and butyl parabens. The preservative is preferably present in the composition in an amount from about 0 to about 3 wt. %, from about 0.1 to about 2 wt. %, and from about 0.2 to about 1 wt. %.

Other components that may be included are film formers, moisturizers, minerals, viscosity and/or rheology modifiers, insect repellents, skin cooling compounds, skin protectants, lubricants, pearls, chromalites, micas, conditioners, anti-allergenics, antimicrobials (e.g., antifungals, antivirals, antibacterials), antiseptics, pharmaceutical agents, photostabilizing agents, surface smoothers, optical diffusers, and exfoliation promoters. Details with respect to these and other suitable cosmetic ingredients can be found in the “International Cosmetic Ingredient Dictionary and Handbook,” 10th Edition (2004), published by the Cosmetic, Toiletry, and Fragrance Association (CTFA), at pp. 2177-2299, which is herein incorporated by reference in its entirety. The amounts of these various substances are those that are conventionally used in the cosmetic or pharmaceutical fields, for example, they can constitute from about 0.01% to about 20% of the total weight of the composition.

The composition can include pH adjusters (e.g., citric acid, ethanolamine, sodium hydroxide, etc.) to be formulated within a wide range of pH levels. In one embodiment, the pH of the topical composition ranges from 1.0 to 13.0. In some embodiments, the pH of the topical composition ranges from 2.0 to 12.0. Other pH ranges suitable for the subject composition include from 3.5 to 7.0, or from 7.0 to 10.5. Suitable pH adjusters such as sodium hydroxide, citric acid and triethanolamine may be added to bring the pH within the desired range.

In certain embodiments, the topical composition may be formulated so that it can be applied, for example, via pen, tube, bottle, brush, stick, sponge, cotton swab, towelette (wipe), sprayer, dropper, hand, or finger.

The topical composition may be formulated in a variety of product forms, such as, for example, a lotion, cream, serum, spray, aerosol, liquid cake, ointment, essence, gel, paste, patch, pencil, powder, towelette, soap or other cleanser, shampoo, conditioner, stick, foam, mousse, elixir or concentrate.

In one embodiment, the composition can be provided in the form of a facial mask. The mask may be a peel-off mask or wash-off mask comprising the composition of the present invention.

The topical composition can be formulated as, for example, a suspension, emulsion, nano-emulsion, hydrogel, multiphase solution, liposomal dispersion, lotion, cream, gel, foam, ointment, paste, spray, conditioner, shampoo, mask, cleanser, micellar water, tonic, makeup (e.g., lipstick, foundation, bronzer, rouge, eyeshadow), and/or after-shave.

The composition can be placed in containers of appropriate size, taking into consideration, for example, the intended use, and the contemplated method of application. Thus, the containers into which the composition is placed may be, for example, from 0.1 gallon to 1,000 gallons or more. The composition may further be placed into smaller containers, such as bottles (e.g., 1.5 oz, 500 ml and 1 liter bottles), for distribution of individual doses of the composition.

### Method of Use

The modified sophorolipids of the present invention may be used in the controlled formation and collapse of foams used in, for example, foods, beverages, pharmaceuticals, personal care products, low-rise medical foams, cosmetics, cleaning products, dental care and biotechnology.

In one embodiment, the modified sophorolipids of the present invention may be useful in the control of foaming in cleaning products. The modified sophorolipids may be incorporated into personal care products and cosmetics to provide stable foam. The modified sophorolipids may be removed through rinsing.

In some embodiments, the composition can be formulated as, e.g., a suspension, an emulsion, a hydrogel, a paste, a multiphase solution, a vesicular dispersion, a liquid, a gel, or a powder, for use in, e.g., a toothpaste, a mouthwash, a mouth and/or throat spray, a lozenge, a breath mint, a pen or tube with a brush, sponge, or nozzle applicator, a dissolvable strip, an adhesive tape for the teeth, a toothpick, a chewing gum, a tongue scraper, a lip balm, a syringe, a nasal aspirator, a dental floss and/or any other oral care product known in the art.

In one embodiment, the modified sophorolipids of the present invention may be useful in applications where it is desirable that the properties of a foam respond to contact with the human body. For example, it may be desirable to alter the stability of a food or beverage foam on exposure to the pH and temperature characteristic of the human mouth, altering the flavour release properties, mouthfeel, viscosity or other properties of the foam. Alternatively, it may be desirable to alter the stability of a dental foam on exposure to the pH and temperature characteristic of the human mouth or the mouth of a particular non-human species, for example to transform a stable and less active stored form of a dental care product into a more active form. Alternatively, it may be desirable to alter the stability of a personal care or cosmetic foam on exposure to the temperature and pH characteristic of human skin, for example to enhance the appearance of a cosmetic or personal care product. In another embodiment, it may be desirable to alter the stability of a pharmaceutical foam on exposure to the temperature and pH characteristic of human skin, for example to enhance skin permeation by a pharmaceutical product.

In some embodiments, the present invention is directed to a method of treating/improving skin conditions with the modified sophorolipid or the composition comprising the modified SLP as described herein, wherein a topical cosmetic composition of the subject invention is applied directly to an area of the skin where such a condition exists.

As used herein, the term “skin condition” encompass any human and animal conditions, disorders, or diseases affecting the epidermis, dermis (including connective tissue, sebaceous glands, and hair follicles), and/or the subcutaneous tissue (hypodermis). Skin conditions that can, in certain embodiments, be treated and/or prevented using compositions, products and methods described herein include, but are not limited to, wounds (including, e.g., burns), scars, acne, blemishes, rosacea, folliculitis, carcinoma, melanoma, perioral dermatitis, cellulitis, carbuncles, photodamage, skin aging (e.g., wrinkles and dryness), age spots, psoriasis, ichthyosis, atopic dermatitis, rashes (including but not limited to erythematosus, macular, papular and/or bullous conditions), chronic wounds, bed sores, keratosis pilaris, sebaceous cysts, vitiligo, melisma, warts, inflammatory dermatoses, allodynia, ectopic dermatitis, telangiectasia, post-inflammatory hyperpigmentation, keratoses, eczema, xerosis, pruritis, lichen planus, nodular prurigo, microbial infection, body odor, scalp conditions and miliaria. Symptoms of skin conditions can include, for example, skin irritation/sensitivity, blemishes and other acneiform symptoms, pigmentation, or loss thereof, flushing, inflammation, wrinkles, dryness, sagging, thickening, scaling, scarring, flaking, rash, hives, blisters, ulcers, peeling, hair loss and other changes in the health, function, and appearance of the skin. In some embodiments, a symptom of a skin condition can also be a skin condition itself.

In some embodiment, the present invention is directed to a method of treating or preventing acne, or acne vulgaris. Acne results from the action of hormones and other impurities at the skin's oil glands and hair follicles. Acne is a systematic inflammatory disease resulting from blockage of sebaceous glands and hair follicles, which can become infected by the bacterium Propionibacterium acnes (P. acnes). Acne as used herein also includes comedonal acne (or “comedones”), which causes bumpy skin, blackheads, and non-inflamed blemishes.

In certain embodiments, the method of treating acne comprises topically administering to a subject an effective amount of the modified sophorolipid or the composition comprising the modified sophorolipid of the subject invention. In other embodiments, the composition further comprises a therapeutic agent selected from, for example, retinoids (e.g., retinol, adapalene, tretinoin, isotretinoin), and alpha hydroxy acids.

In some embodiments, the modified sophorolipids alone may be topically applied to the affected area of the skin or is prepared into a topical skin care composition comprising additional ingredients. As discussed above, the compositions comprising the modified sophorolipids may be provided in a variety of forms including, but are not limited to, creams, lotions, masks, powders, and cleansers. The compositions according to the invention may be used in a method to remove makeup and other impurities from the face and skin.

In further embodiments, the present invention is directed to a method of treating or preventing a variety of other skin conditions, including hyperpigmentation, wrinkles, and skin looseness. The method comprises topically administering a modified sophorolipid or a composition comprising the modified SLP. In a specific embodiment, the composition further comprises salicylic acid, succinic acid, amino acids, retinol and/or alpha hydroxy acids.

In certain embodiments, the topical therapeutic composition is applied, e.g., every other day, once daily, up to ten times daily. In some embodiments, the topical composition is applied every other day, once daily, up to ten times daily, for an indefinite period of time, e.g., for at least one, two, three weeks, or longer, until the skin condition is healed and/or improved.

In certain embodiments, the composition is rubbed into the skin so that the composition is absorbed therein. In some embodiments, the composition can be applied to the skin for an amount of time (e.g., in the form of a mask) and then rinsed from the skin using, for example, water.

In certain embodiments, skin of the subject's face and neck is treated according to the present methods. In some embodiments, any part of the subject's skin can be treated, including, for example, ears, chest, shoulders, back, arms, underarms, hands, stomach, buttocks, legs and feet.

In some embodiments, the subject invention provides methods of enhancing and/or maintaining the oral health of a mammalian subject, wherein a composition of the subject invention is applied to one or more surfaces of the subject's mouth, lips or oral cavity. The one or more surfaces can include, for example, the subject's teeth, gums, tongue, palate, floor, roof, throat, pharynx, tonsils, cheeks, lips (interior and exterior), epiglottis, and any of the tissues or passageways connected thereto.

In some embodiments, the method further comprises rinsing the composition from the one or more surfaces of the mouth, lips or oral cavity. This can be performed with, for example, water or mouth wash.

In preferred embodiments, the method can be used to treat and/or prevent plaque, biofilm and/or tartar formation; halitosis; oral infections/abscesses; mouth sores, including ulcers, cold sores and/or canker sores; stained teeth; thrush; oral cancers; gingivitis; tooth decay; periodontal disease; caries; and/or any other oral health condition caused by the presence of bacteria, fungi and/or viruses, such as, e.g., inflammatory conditions, tonsillitis, pharyngitis, laryngitis, glossitis, stomatitis and others.

In certain embodiments, the method can be used for reducing the number of undesirable microorganisms present in a subject's oral cavity, thus allowing for an increase in beneficial and/or commensal microflora. For example, the method can be used for controlling microbes that cause plaque and tartar buildup on teeth, halitosis, and those that lead to periodontal disease and/or cavities.

In some embodiments, the method can be used to enhance the healing (e.g., reduce the healing time) of open sores and wounds in the oral cavity, for example, canker sores, cold sores, cuts, bite wounds, burns from hot foods or beverages, surgical incisions and others caused by tissue trauma.

In one embodiment a method is provided for increasing the penetration and/or bioavailability of a dental and/or oral health pharmaceutical compound, wherein a composition of the subject invention is applied with the pharmaceutical compound as an adjuvant. For example, the composition can be administered as an adjuvant for an analgesic, or a gel used for treating canker sores or cold sores. Advantageously, the method can allow for reduced concentrations of the pharmaceutical compound to be administered to the subject in need thereof while remaining therapeutically effective.

### EXAMPLES

Following are exemplary compounds of the present invention, which are offered by way of illustration and are not intended to limit the invention.

The foaming power and the foam stability can be determined by, for example, the Ross-Miles method. Examples of the modified SLP of the present invention includes, for example, compounds in Table 1.

TABLE 1
Exemplary compounds.

| Com- | |
|---|---|
| pound | Structure |
| 1 | 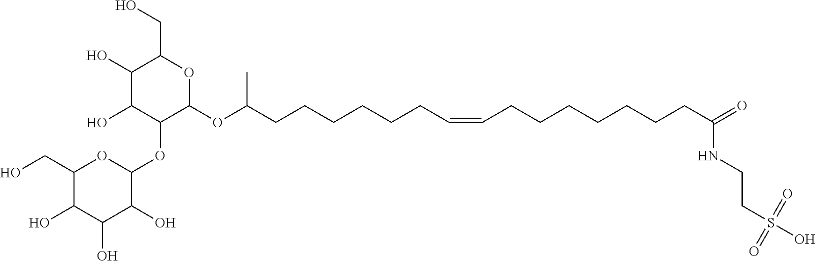 [SMILES: CC(CCCCCC/C=C\CCCCCCCC(=O)NCCS(=O)(=O)O)OC1OC(CO)C(O)C(O)C1OC1OC(CO)C(O)C(O)C1O] |
| 2 | 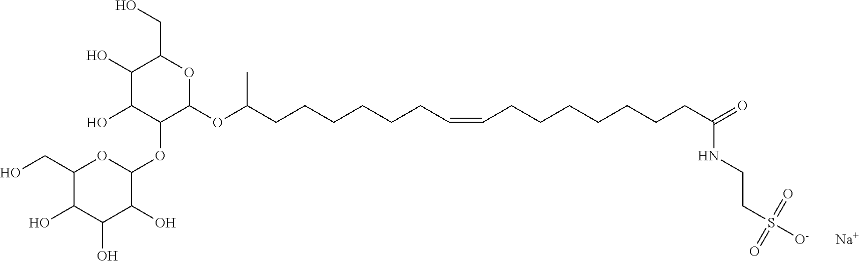 [SMILES: CC(CCCCCC/C=C\CCCCCCCC(=O)NCCS(=O)(=O)[O-])OC1OC(CO)C(O)C(O)C1OC1OC(CO)C(O)C(O)C1O ; [Na+]] |
| 3 | 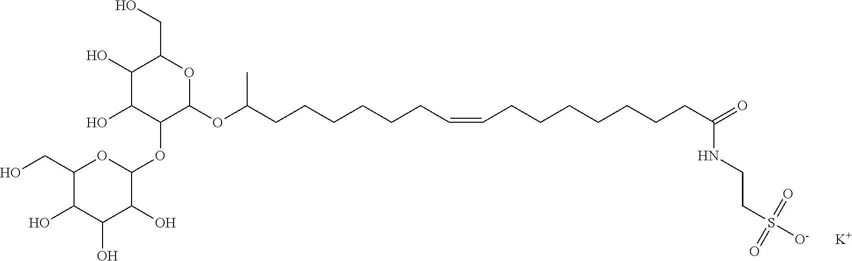 [SMILES: CC(CCCCCC/C=C\CCCCCCCC(=O)NCCS(=O)(=O)[O-])OC1OC(CO)C(O)C(O)C1OC1OC(CO)C(O)C(O)C1O ; [K+]] |
| 4 | 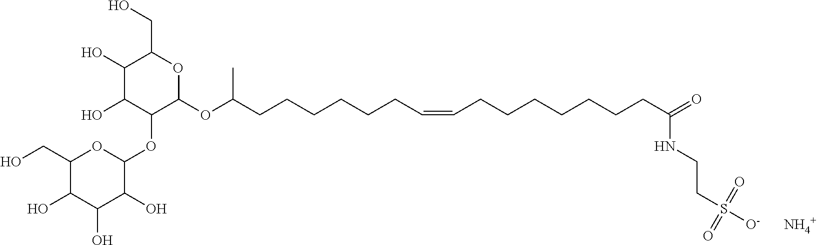 [SMILES: CC(CCCCCC/C=C\CCCCCCCC(=O)NCCS(=O)(=O)[O-])OC1OC(CO)C(O)C(O)C1OC1OC(CO)C(O)C(O)C1O ; [NH4+]] |
| 5 | 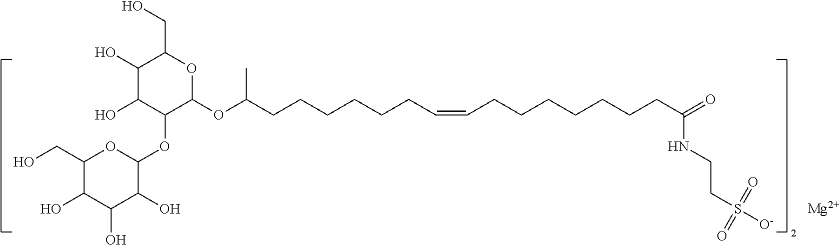 |
| 6 | 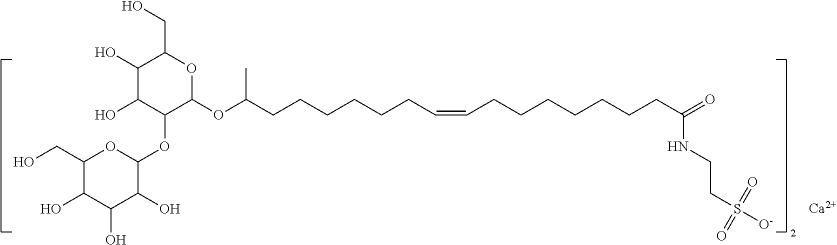 |
| 7 | 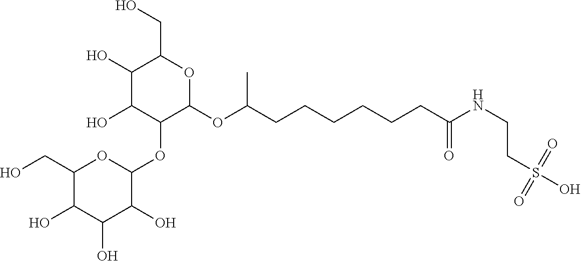 [SMILES: CC(CCCCCCC(=O)NCCS(=O)(=O)O)OC1OC(CO)C(O)C(O)C1OC1OC(CO)C(O)C(O)C1O] |
| 8 | 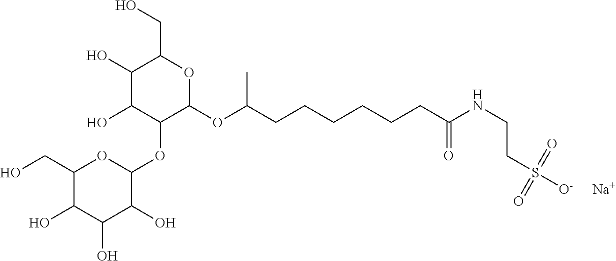 [SMILES: CC(CCCCCCC(=O)NCCS(=O)(=O)[O-])OC1OC(CO)C(O)C(O)C1OC1OC(CO)C(O)C(O)C1O ; [Na+]] |
| 9 | 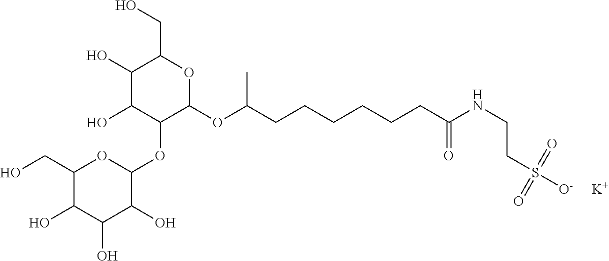 [SMILES: CC(CCCCCCC(=O)NCCS(=O)(=O)[O-])OC1OC(CO)C(O)C(O)C1OC1OC(CO)C(O)C(O)C1O ; [K+]] |
| 10 | 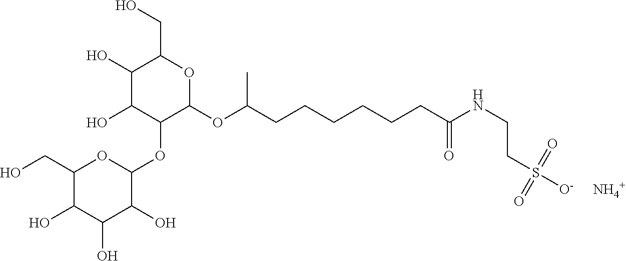 [SMILES: CC(CCCCCCC(=O)NCCS(=O)(=O)[O-])OC1OC(CO)C(O)C(O)C1OC1OC(CO)C(O)C(O)C1O ; [NH4+]] |
| 11 | 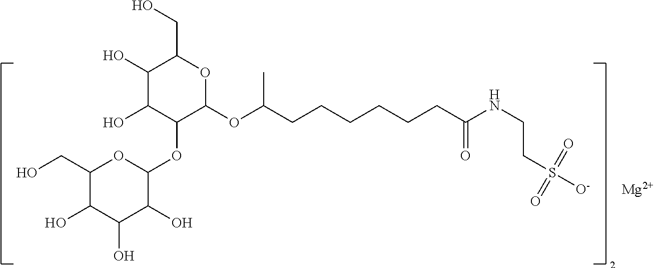 |
| 12 | 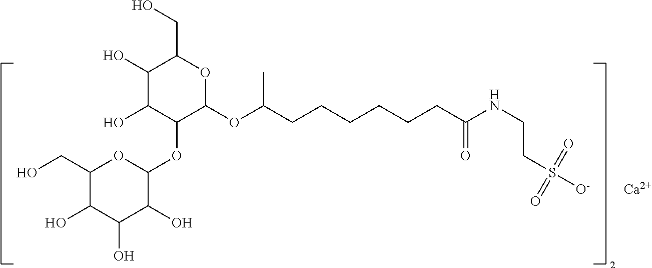 |
| 13 | 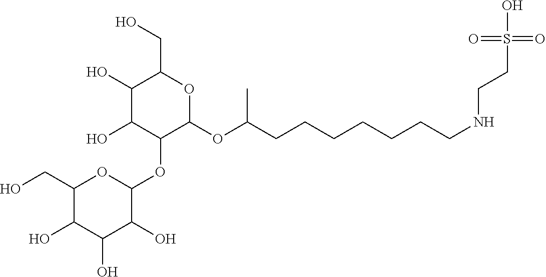 [SMILES: CC(CCCCCCCNCCS(=O)(=O)O)OC1OC(CO)C(O)C(O)C1OC1OC(CO)C(O)C(O)C1O] |
| 14 | 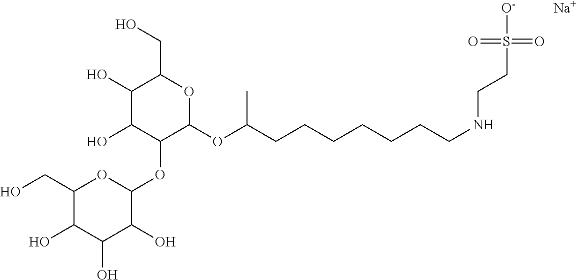 [SMILES: CC(CCCCCCCNCCS(=O)(=O)[O-])OC1OC(CO)C(O)C(O)C1OC1OC(CO)C(O)C(O)C1O ; [Na+]] |
| 15 | 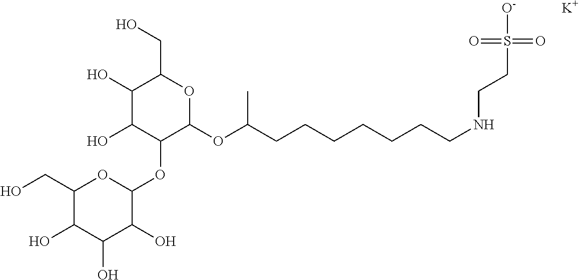 [SMILES: CC(CCCCCCCNCCS(=O)(=O)[O-])OC1OC(CO)C(O)C(O)C1OC1OC(CO)C(O)C(O)C1O ; [K+]] |
| 16 | 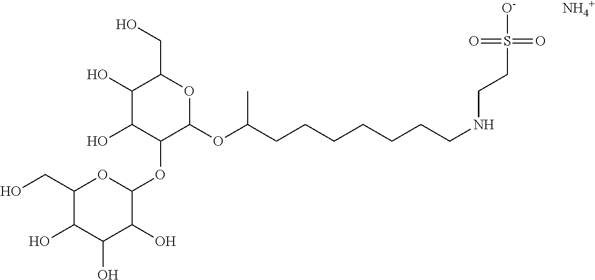 [SMILES: CC(CCCCCCCNCCS(=O)(=O)[O-])OC1OC(CO)C(O)C(O)C1OC1OC(CO)C(O)C(O)C1O ; [NH4+]] |
| 17 | 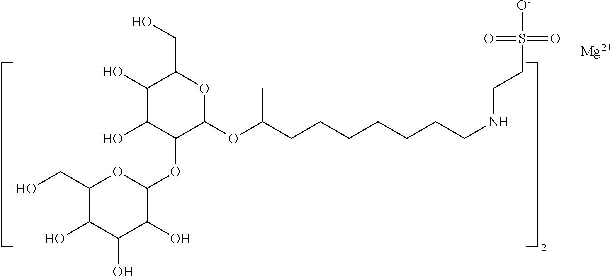 |
| 18 | 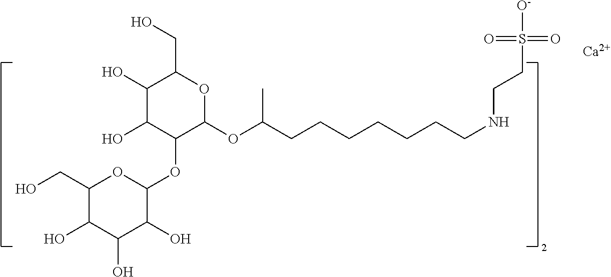 |
| 19 | 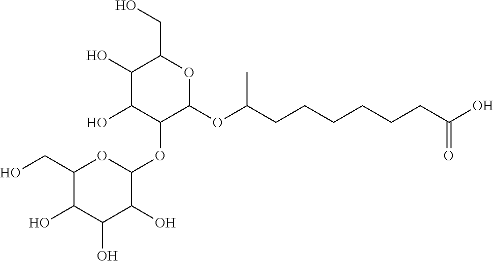 [SMILES: CC(CCCCCCC(=O)O)OC1OC(CO)C(O)C(O)C1OC1OC(CO)C(O)C(O)C1O] |
| 20 | 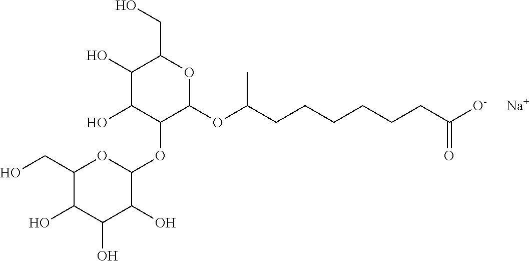 [SMILES: CC(CCCCCCC(=O)[O-])OC1OC(CO)C(O)C(O)C1OC1OC(CO)C(O)C(O)C1O ; [Na+]] |
| 21 | 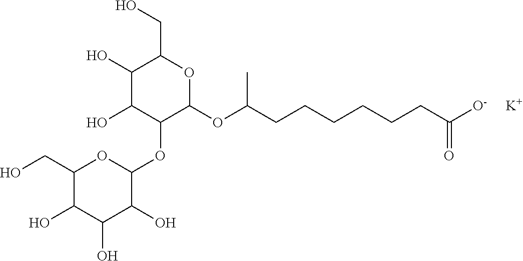 [SMILES: CC(CCCCCCC(=O)[O-])OC1OC(CO)C(O)C(O)C1OC1OC(CO)C(O)C(O)C1O ; [K+]] |
| 22 | 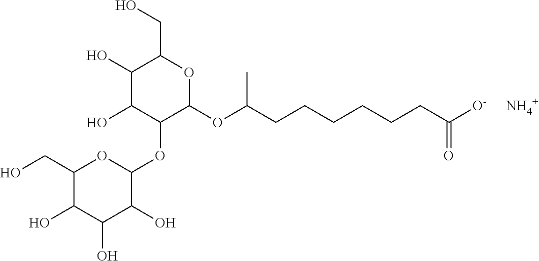 [SMILES: CC(CCCCCCC(=O)[O-])OC1OC(CO)C(O)C(O)C1OC1OC(CO)C(O)C(O)C1O ; [NH4+]] |
| 23 | 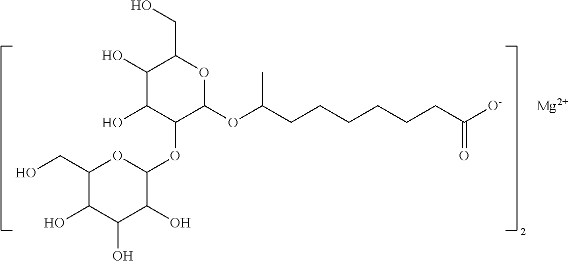 |
| 24 | 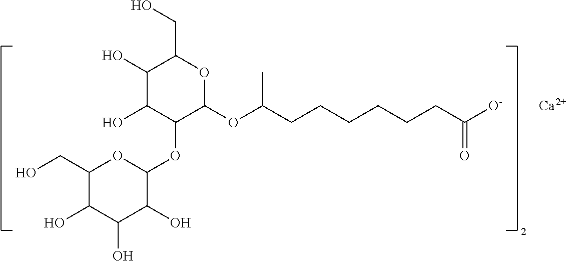 |

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application

1. A compound having a structure selected from: 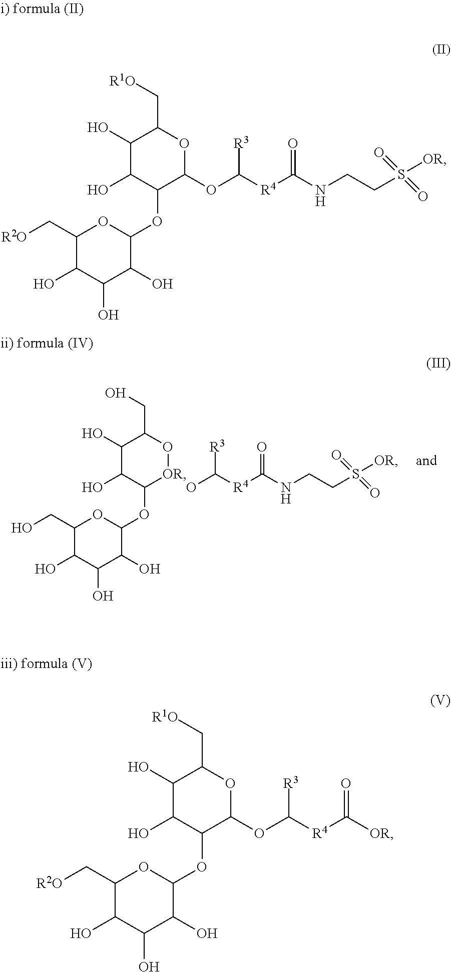
wherein R¹ and R² are each independently selected from hydrogen, alkyl, substituted alkyl, acyl, substituted acyl, alkenyl, and substituted alkenyl; R³ is hydrogen or alkyl; R⁴ is alkane, alkylene, substituted alkylene, alkenylene or substituted alkenylene; and R is hydrogen or a cation selected from Na⁺, K⁺, Li⁺, Cs⁺, Rb⁺, Ca²⁺, Mg²⁺ and NH₄⁺.

2. The compound of claim 1, wherein R¹ and R² are hydrogen; and R³ is methyl.

3. The compound of claim 1, wherein R⁴ is C5-C15 alkylene, or C5-C15 alkenylene.

4. The compound of claim 1, which has a structure of: 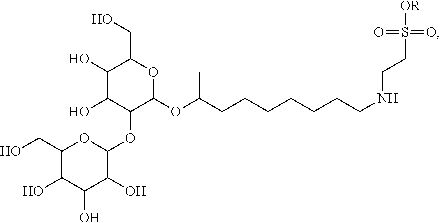
wherein R is hydrogen or a cation selected from Na⁺, K⁺, Li⁺, Cs⁺, Rb⁺, Ca²⁺, Mg²⁺ and NH₄⁺.

5. The compound of claim 1, which has a structure of: 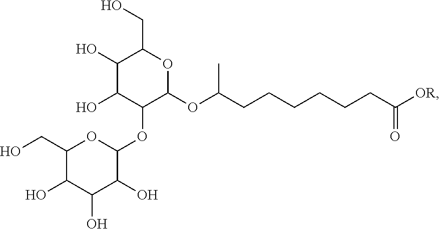
wherein R is hydrogen or a cation selected from Na⁺, K⁺, Li⁺, Cs⁺, Rb⁺, Ca²⁺, Mg²⁺ and NH₄⁺.

6. The compound of claim 1, which has a structure of formula (III): 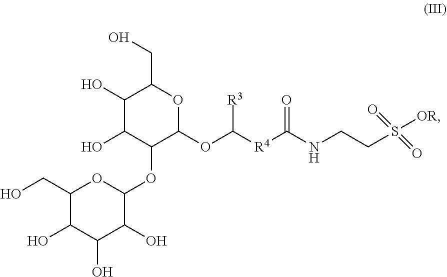
wherein R is hydrogen or a cation selected from Na⁺, K⁺, Li⁺, Cs⁺, Rb⁺, Ca²⁺, Mg²⁺ and NH₄⁺.

7. The compound of claim 1, which has a structure of: 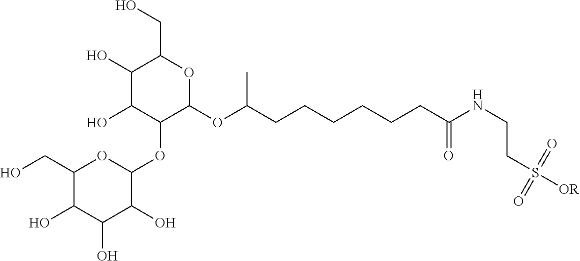
wherein R is hydrogen or a cation selected from Na⁺, K⁺, Li⁺, Cs⁺, Rb⁺, Ca²⁺, Mg²⁺ and NH₄⁺.

8. The compound of claim 1, which has a structure of: 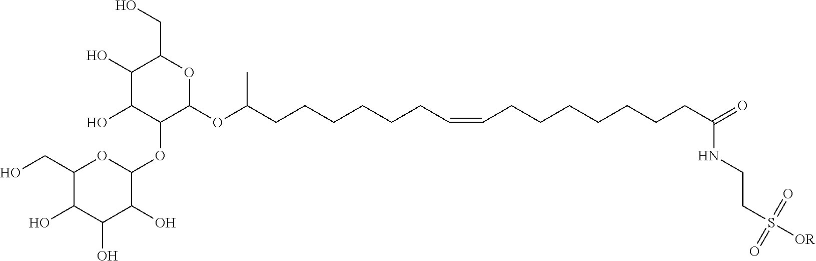
wherein R is hydrogen or a cation selected from Na⁺, K⁺, Li⁺, Cs⁺, Rb⁺, Ca²⁺, Mg²⁺ and NH₄⁺.

9. A composition comprising the compound of claim 1.

10. The composition of claim 9, further comprising a pharmaceutically or cosmetically acceptable carrier.

11. The composition of claim 9, further comprising one or more ingredients selected from viscosity modifiers, skin conditioners, anti-aging components, exfoliating agents, antioxidants, fragrances, and preservatives.

12. The composition of claim 9, wherein the composition is formulated as a suspension, emulsion, nano-emulsion, hydrogel, multiphase solution, liposomal dispersion, lotion, cream, gel, foam, ointment, paste, spray, conditioner, shampoo, cleanser, micellar water, tonic, toothpaste, mouthwash or makeup.

13. A method for improving the foaming performance of a sophorolipid, the method comprising providing the sophorolipid, and functionalizing the sophorolipid by covalently linking to taurine through an amide bond or amine bond.

14. The method of claim 13, wherein the sophorolipid has a structure of formula (I): 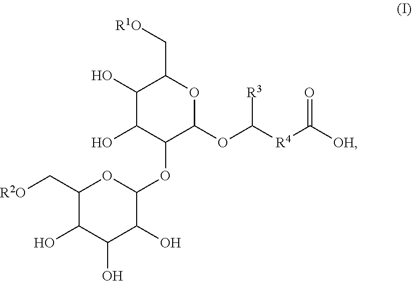
wherein R¹ and R² are each independently selected from hydrogen, alkyl, substituted alkyl, acyl, substituted acyl, alkenyl, and substituted alkenyl; R³ is hydrogen or alkyl; and R⁴ is alkane, alkylene, substituted alkylene, alkenylene or substituted alkenylene.

15. The method of claim 13, wherein R¹ and R² are hydrogen; and R³ is methyl.

16. The method of claim 13, wherein R⁴ is C5-C15 alkane, C5-C15 alkylene, or C5-C15 alkenylene.

17. A method for producing the composition of claim 9, the method comprising providing a sophorolipid, functionalizing the sophorolipid by covalently linking to taurine through an amide bond or amine bond, and mixing the functionalized sophorolipid with a solvent.

18. A method for improving a skin condition of a subject, wherein the method applying the composition of claim 9 to an area of the skin of the subject.

19. A method for enhancing and/or maintaining the oral health of a subject, wherein the method comprises applying the composition of claim 9 to a surface of the subject's mouth, lips or oral cavity.

20. A method for cleaning, enhancing and/or maintaining heathy hair of a subject, wherein the method comprises applying the composition of claim 9 to the hair of the subject.
